(19) United States
(12) Patent Application Publication
(10) Pub. No.: US 2020/0277115 A1
(43) Pub. Date: Sep. 3, 2020

(54) Defect-Indicating Gasket for Sterilization Container

(71) Applicant: O&M Halyard, Inc., Mechanicsville, VA (US)

(72) Inventor: Namita A. Mithani, Alpharetta, GA (US)

(21) Appl. No.: 16/802,043

(22) Filed: Feb. 26, 2020

Related U.S. Application Data
(60) Provisional application No. 62/811,613, filed on Feb. 28, 2019.

(51) Int. Cl. B65D 55/02; B65D 43/02; B65D 53/02; B65D 25/28; A61L 2/28

(52) U.S. Cl. CPC B65D 55/026; B65D 43/0202; B65D 53/02; A61L 2202/24; A61L 2/28; A61L 2202/182; B65D 25/2838

(57) ABSTRACT

A sterilization packaging system with features for sealing a volume against an ingress of contaminants is provided. Such features include a sealing gasket and a sheet of filter material. The gasket seals a lid to a seal rim of a container base of the sterilization packaging system. The gasket can enable quick identification of punctures, holes, tears, etc. so that the user notices immediately that the seal has been breached and that the contents of the sterilization packaging have been exposed to the outside environment.

### RELATED APPLICATIONS

The present application claims priority to U.S. Provisional Application Ser. No. 62/811,613, filed on Feb. 28, 2019, which is incorporated herein in its entirety by reference thereto.

### COLOR DRAWINGS

The patent or application file contains a least one drawing executed in color. Copies of this patent or patent application publication with color drawing(s) will be provided by the Office upon request and payment of the necessary fee.

### FIELD OF THE INVENTION

The subject matter of the present disclosure relates generally to sterilization packaging and sterilization packaging systems.

### BACKGROUND

Personnel in the Central Service Room (CSR) or the Sterile Processing Department (SPD) of hospitals are commonly charged with the responsibility of packaging surgical supplies into various types of sterilization packaging systems and sterilizing the systems to ensure that the sterility of the packaged contents is maintained from sterilization to the point of reuse. Several activities are involved in the task of preparing medical supplies (e.g., devices, accessories, components, etc.) that are contained in the sterilization packaging system for delivery to the operating room, cardiac catheterization lab, emergency room, labor and delivery room, intensive care unit, pediatric care unit, specialized burn care units, and other surgical or medical units.

Much of the surgical instruments and supplies used in the operating room are reusable. These supplies typically include such things as clamps, scalpel blade handles, retractors, forceps, scissors, surgeon's towels, basins, and the like. All of these supplies must be collected after each procedure, decontaminated, washed, placed in a sterilization packaging system, and sterilized before they can be used again in another procedure. The sterilization packaging systems used must be of the size and shape to accommodate the items to be sterilized, must be compatible with and withstand the physical conditions of the sterilization process, and must be capable of maintaining the sterility of their contents post-sterilization.

Typical means of sterilizing instruments include, among others, autoclaving with pre-vacuum and gravity steam, exposure to ethylene oxide gas, and exposure to hydrogen peroxide plasma or vaporized hydrogen peroxide. After the packaging system and its contents have been sterilized, the sterilization packaging system typically is stored until it is needed for a surgical or medical intervention procedure, at which time it is transported to the operating room, cardiac catheterization lab, emergency room, labor and delivery room, intensive care unit, pediatric care unit, specialized burn care units, or any other surgical or medical unit, or it can be stored in an environmentally controlled room until it is needed by the medical staff for utilization in a surgical or medical intervention procedure.

Common sterilization packaging systems include sealable pouches, sterilization wraps, and rigid containers. Although each of these systems has some advantage compared to other systems, each of these typical packaging systems also has drawbacks. As an example, using a sterilization wrap to package items to be sterilized in a certain prescribed manner will permit the entry of sterilizing vapor/gas or other medium to sterilize the contents of the wrapped package while denying the ingress of contaminants such as bacteria and other infection causing materials or their vehicles after sterilization. As such, sterilization wraps generally provide a consistent barrier against the ingress of contaminants. However, during storage and transfer to the operating room, the wrapped package may be handled several different times; each time the wrapped package is handled, there is a potential that the sterile nature of the package contents can be compromised, e.g., by a tear, cut, or other breach of the wrapping material, which can occur due to over handling or careless manipulation of the wrapped package.

As another example, sterilization containers—such as, e.g., a metal box and a rigid top or lid that closes the metal box—also can permit the entry of sterilizing medium while denying the ingress of contaminants after sterilization. Unlike sterilization wraps, rigid sterilization containers usually avoid tears, cuts, and the like that can compromise the sterilized contents of the container. However, typical rigid sterilization containers are complex packaging systems, including several parts that must be precisely assembled to prevent compromising the contents of the container after sterilization. For example, rigid sterilization containers often require a metal box, a lid, a filter attached to the metal box and/or lid by various means such as an elastic band around the perimeter of the filter or multiple hook and loop fasteners or retainer plates around the filter, and a gasket to form a seal between the metal box and the lid.

In particular, the gasket that is used to seal the container lid to the container base is typically integrated into the container lid, which is reusable, where such reusability can lead to the formation defects in the gasket. For instance, over time the gasket can experience wear and tear due to thermo-mechanical or physical stresses associated with repeated sterilization, disinfection, and/or mechanical washing. In addition, the number of sterilization cycles to which a gasket is subjected is not easily tracked and there is no indication for when a gasket needs to be replaced. This wear and tear can result in compromised quality of the gasket, which may have cuts and tears which are very small or difficult to notice. The compromised quality of the gasket therefore results in a compromised seal of the container assembly.

Moreover, the gaskets and locking mechanisms for the retention plate can degrade over time, which can also increase the potential for bacterial ingress and contamination.

Consequently, there is a need for a sterilization packaging system that overcomes the shortcomings of known packaging systems. In particular, a gasket that enables quick identification of punctures, holes, tears, etc. so that the user notices immediately that the seal has been breached and that the contents of the sterilization packaging have been exposed to the outside environment would also be useful.

### SUMMARY

The present invention is directed to sealing assembly for a sterilization packaging system. The sealing assembly includes a gasket having an outer edge, an inner edge, and an upper surface and a lower surface each extending from the outer edge to the inner edge. The gasket includes an outer layer and an inner core. The outer layer includes a first material compounded with a first colored pigment, and the inner core includes a second material compounded with a second colored pigment. A sufficient level of contrast exists between the outer layer and the inner core to detect a breach of the outer layer.

In one particular embodiment, a breach of the outer layer can expose the second colored pigment of the inner core to facilitate detection of the breach.

In one more embodiment, the outer layer is darker than the inner core, wherein the outer layer and the inner core exhibit a ΔE* color difference greater than about 2.5 as determined according to the Commission Internationale de l'Eclairage (CIE) 1976 standard.

In yet another embodiment, the inner core can exhibit a saturation level of greater than about 25%.

In still another embodiment, the inner core can exhibit a value level of greater than about 25%.

In a further embodiment, the second material can be less compressible and more rigid than the first material.

In an additional embodiment, the first material and the second material can be made from the same polymer. Further, the first material and the second material can have different durometers, wherein the first material has a lower durometer than the second material.

In one more embodiment, the first material and the second material can be made from polyurethane, silicone, polyvulcanate, polyvinylidene chloride (PVDC), polytetrafluoroethylene (PTFE), polysulphones, crosslinked elastomers, or a combination thereof.

The present invention is additionally directed to a sterilization packaging system having a volume for containing items to be sterilized. The sterilization packaging system includes a lid having an upper surface defining a perimeter and a lip extending downward from the perimeter; a base having a lower surface, a first sidewall, a second sidewall, a third sidewall, and a fourth sidewall extending from the lower surface, wherein the first sidewall, the second sidewall, the third sidewall, and the fourth sidewall terminate at a seal rim defining a perimeter of an opening of the base; a filter; and a sealing assembly, wherein the sealing assembly is configured to seal the lid to the seal rim of the base. The sealing assembly includes a gasket having an outer edge, an inner edge, and an upper surface and a lower surface each extending from the outer edge to the inner edge. The gasket includes an outer layer and an inner core. The outer layer includes a first material compounded with a first colored pigment, and the inner core includes a second material compounded with a second colored pigment, and a sufficient level of contrast exists between the outer layer and the inner core to detect a breach of the outer layer.

In one particular embodiment of the sterilization packaging system, a breach of the outer layer of the gasket can expose the second colored pigment of the inner core of the gasket to facilitate detection of the breach.

In another embodiment, the outer layer of the gasket can be darker than the inner core of the gasket, wherein the outer layer of the gasket and the inner core of the gasket exhibit a ΔE* color difference greater than about 2.5 as determined according to the Commission Internationale de l'Eclairage (CIE) 1976 standard.

In a further embodiment, the inner core of the gasket can exhibit a saturation level of greater than about 25%.

In an additional embodiment, the inner core of the gasket can exhibit a value level of greater than about 25%.

In still another embodiment, the second material of the gasket can be less compressible and more rigid than the first material of the gasket.

In yet another embodiment, the first material of the gasket and the second material of the gasket can be made from the same polymer. Further, the first material of the gasket and the second material of the gasket can have different durometers, wherein the first material has a lower durometer than the second material.

In an additional embodiment, the first material and the second material can be made from polyurethane, silicone, polyvulcanate, polyvinylidene chloride (PVDC), polytetrafluoroethylene (PTFE), polysulphones, crosslinked elastomers, or a combination thereof.

In a further embodiment, the sealing assembly can be configured to provide a continuous sealing interface between the lid and the base.

In still another embodiment, the sealing assembly and the filter can be disposable, further wherein the lid and the base can be reusable.

In an additional embodiment, the sealing assembly can be reusable and the filter can be disposable, further wherein the lid and the base can be reusable.

These and other features, aspects, and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

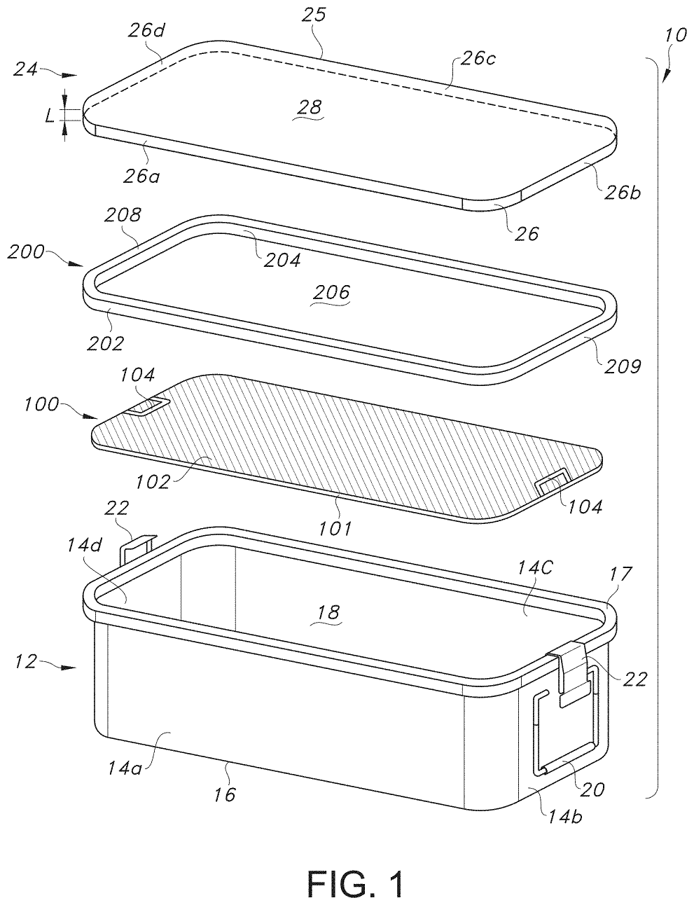

FIG. 1 illustrates an exploded view of an embodiment of the sterilization packaging system according to the present invention;

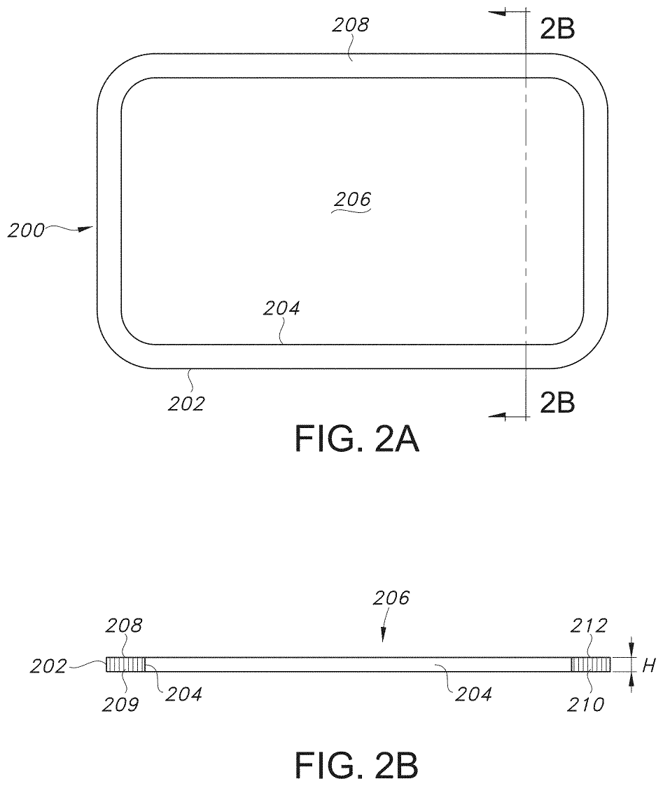

FIG. 2A illustrates a top view of the gasket of FIG. 1;

FIG. 2B illustrates a cross-sectional view of the gasket of FIG. 2A taken along the line 2-2 showing the high contrast between the inner core and outer layer of the gasket to facilitate detection of a breach;

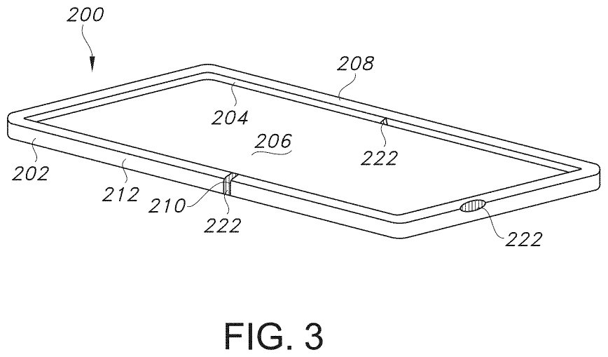

FIG. 3 illustrates a perspective view of the gasket of FIG. 2A having various breaches in the outer layer detected by the high contrast between the inner core and outer layer of the gasket;

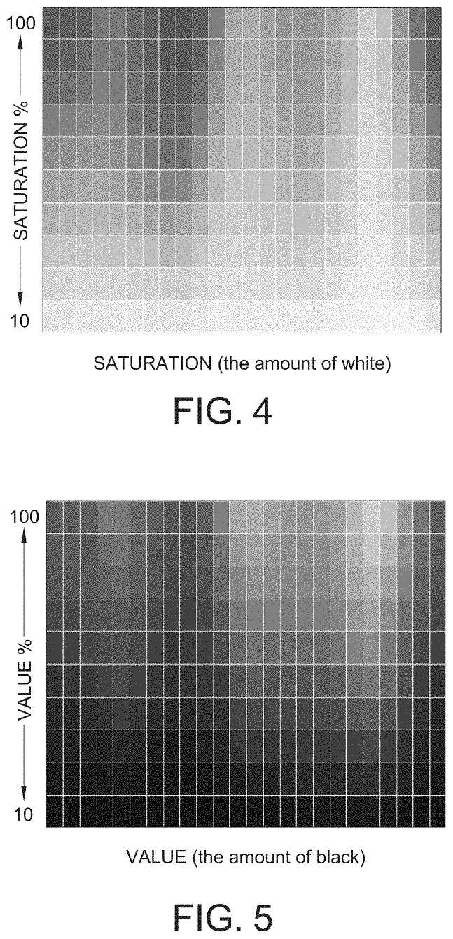

FIG. 4 is a chart illustrating various levels of color saturation; and

FIG. 5 is a chart illustrating various levels of color value.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

Described herein is a sterilization packaging system and components thereof suitable for use in a variety of procedures for containing, sterilizing, storing, and using sterilized items such as surgical supplies. While described in conjunction with its use in hospital and surgical room procedures, the present subject matter is intended for use wherever there is a need for sterilized materials. Consequently, the following description should not be considered a limitation as to the scope of use of the present subject matter.

Generally speaking, the present invention is directed to sealing assembly for a sterilization packaging system. The sealing assembly includes a gasket having an outer edge, an inner edge, and an upper surface and a lower surface each extending from the outer edge to the inner edge. The gasket includes an outer layer and an inner core. The outer layer includes a first material compounded with a first colored pigment, and the inner core includes a second material compounded with a second colored pigment. A sufficient level of contrast exists between the outer layer and the inner core to detect a breach of the outer layer. The present invention is also directed to a sterilization packaging system that includes the sealing assembly. Because of the specific components and arrangement of the sealing assembly and sterilization packaging system, a user can more easily and quickly identify punctures, holes, tears, etc. indicating that the seal has been breached and that the contents of the sterilization packaging have been exposed to the outside environment or contamination.

FIG. 1 provides a perspective view of a sterilization packaging system according to an exemplary embodiment of the present invention. In the depicted embodiment, sterilization packaging system 10 includes a container base 12 and a lid 24 together defining a volume for containing one or more items to be sterilized. The base 12 includes a lower surface 16, a first sidewall 14a, a second sidewall 14b, a third sidewall 14c, and a fourth side wall 14d extending from the lower surface 16.

One or more handles 20 can be present on one or more of the sidewalls. Although FIG. 1 only shows a handle 20 present on the second sidewall 14b, the present invention also contemplates a handle 20 on the fourth sidewall 14d in the embodiment shown in FIG. 1 for balanced carrying of the sterilization packaging system 10. The base 12 also includes an upper opening 18 to provide access to the volume in which items to be sterilized can be placed. The first sidewall 14a, second sidewall 14b, third sidewall 14c, and fourth sidewall 14d terminate in a seal rim 17 extending around a periphery of the upper opening 18 of the base 12. The base 12 can have a generally rectangular shape, for example a generally rectangular shape having rounded corners as shown in FIG. 1, or any other suitable shape for a container configured to contain surgical tools.

Referring again to FIG. 1, the lid 24 includes an upper surface 28 having a perimeter 25, where a lip 26 extends downward from the perimeter 25 towards the base 12. The lip 26 includes a first side 26a, a second side 26b, a third side 26c, and a fourth side 26d which correspond to and are configured to extend around the sides 14a, 14b, 14c, 14d of the base 12, respectively. The lip 24 can have a height Las shown in FIG. 1 that is configured to extend around or over the gasket 200, described in further detail below. As shown in FIG. 1, the lid 24 may be sealed to the base 12 via a conventional vertical-swinging latch 22 or any other suitable locking mechanism. The base 12 and the lid 24 can be reusable and can be formed from a rigid material such stainless steel, anodized aluminum, polyetheretherketone (PEEK), polyaryletherketone, polyphenylsulphone (PPSU), polysulphone (PSU), filled PPSU, and filled PSU.

Once sealed (not shown), the sealed sterilization packaging system 10 can then be transferred to sterilizing equipment and exposed to sterilization conditions as generally known in the art. Such sterilization conditions can include, for example, steam, ethylene oxide, or hydrogen peroxide plasma sterilization conditions. Sterilization conditions are the conditions present during a particular sterilization methodology utilized that substantially kills or completely destroys bacteria and other infectious organisms in an industrial or medical product to the desirable sterility assurance level (e.g., 0-6 log reduction for terminal sterilization).

Referring again to FIG. 1, specifically regarding the sealing of the sterilization packaging system 10, the lid 24 can be sealed to the base 12 via a gasket 200 that is engaged and compressed between the lid 24 and the seal rim 17 of the base 12 via the latches 22 or other locking mechanism. The sterilization packaging system 10 can additionally include a filter 100. The filter 100 can cover the entire opening 18 of the base 12 by extending from the first side 14a to the third side 14c and from the second side 14b to the fourth side 14d of the base 12. The gasket 200 and filter 100 together can seal the base 12 and prevent the ingress of contaminants such as, e.g., bacteria and other infection causing materials or their vehicles, into the volume of the base 12. As shown in FIG. 1, the gasket 200 can be positioned between the lid 24 and the seal rim 17 of the base 12 to hold the filter 100 in place when the sterilization packaging system 10 is assembled. In other embodiments (not shown), the gasket 200 can extend within a groove defined within the lip 26 of the lid 24, or in still other embodiments, the gasket 200 can extend within a groove defined within the seal rim 17 of the base 12.

The filter 100 can be made from a single sheet of filter material 102. The sheet of filter material 102 can be made from a number of materials and, generally, may be made of a material from one of two main classes, reusables and disposables. Reusables are materials that, as the name suggests, can be reused, typically by washing or some other form of cleaning. Disposables, on the other hand, usually are one-use items that are discarded or recycled after their initial use. Generally, cloth, linen, or other woven materials fall into the reusable category while disposables normally include nonwoven materials made from either or both natural and synthetic fibers such as paper, fibrous polymeric nonwovens, and films (e.g., PTFE porous films or membranes), which are capable of passing sterilants and retarding transmission of bacteria and other contaminants.

Nonwoven sterilization materials present several advantages due to their barrier properties, economics, and consistent quality. The nonwoven materials can be made from a variety of processes including, but not limited to, air laying processes, wet laid processes, hydroentangling processes, spunbonding, meltblowing, staple fiber carding and bonding, and solution spinning. The fibers themselves can be made from a variety of both natural and synthetic materials including, but not limited to, cellulose, rayon, nylon, polyesters, polyolefins, and many other materials. The fibers may be relatively short, staple length fibers, typically less than three inches, or longer and substantially more continuous fibers such as are produced by spunbonding and meltblowing processes. Whatever materials are chosen, the resultant sheet of filter material 102 must be compatible with the particular sterilization technique being used and must also provide both strength and barrier properties to maintain the sterile nature of the contents of the sterilization packaging system 10 until use. In the illustrated embodiment shown in FIG. 1, the sheet of filter material 102 can be a transparent breathable film, a translucent or opaque material, such as, e.g., a translucent breathable film, a SMS material (described below), or the like.

For example, the sheet of filter material 102 may be a spunbonded-meltblown-spunbonded material is made from three separate layers that are laminated to one another. The method of making these layers is known and described in U.S. Pat. No. 4,041,203 to Brock, et al., which is incorporated herein in its entirety by reference. The material of Brock, et al. is a three layer laminate of spunbonded-meltblown-spunbonded layers that is also commonly referred to by the acronym “SMS.” The two outer layers of SMS are a spunbonded material made from extruded polyolefin fibers, or filaments, laid down in a random pattern and then bonded to one another. The inner layer is a meltblown layer also made from extruded polyolefin fibers generally of a smaller diameter than the fibers in the spunbonded layers. As a result, the meltblown layer provides increased barrier properties due to its fine fiber structure, which permits the sterilizing agent to pass through the fabric while preventing passage of bacteria and other contaminants. Conversely, the two outer spunbonded layers provide a greater portion of the strength factor in the overall laminate. The laminate may be prepared using an intermittent bond pattern that is preferably employed with the pattern being substantially regularly repeating over the surface of the laminate. The pattern is selected such that the bonds may occupy about 5% to about 50% of the surface area of the laminate. Desirably, the bonds may occupy about 10% to about 30% of the surface area of the laminate. In an exemplary embodiment, the sheet of filter material 102 can be made from a SMS material, but it is to be understood that the sheet of filter material 102 also may be made from other suitable materials. In one particular embodiment, the SMS material of the sheet of filter material 102 can be polypropylene.

As shown in FIG. 1, the filter 100 can be made from a sheet of filter material 102 that is large enough to cover the entire opening 18 of the base 12, i.e. extending from the first sidewall 14a to the third sidewall 14c and from the second sidewall 14b to the fourth sidewall 14d. Although not required, to secure the filter 100 to the base 12, the filter 100 can include an attachment mechanism (not shown). The attachment mechanism of the filter 100 can be located around or on the perimeter 101 of the sheet of filter material 102. The attachment mechanism can be any suitable means to secure the perimeter 101 of the filter 100 to the base 12, e.g. retainer plates as are well known in the art of rigid sterilization containers, or other means. In one embodiment, the filter 100 can additionally include pull-tabs 104 to facilitate aseptic removal of the filter 100 once the lid 24 is removed to expose the sterilized contents contained within the packaging system 10 in the operating room, cardiac catheterization lab, emergency room, labor and delivery room, intensive care unit, pediatric care unit, specialized burn care units, or any other surgical or medical unit.

Referring now to FIGS. 1 and 2A-B, various features of the gasket 200 are shown. The gasket 200 is configured to provide a continuous sealing interface between the lid 24 and base 12 of the sterilization packaging system 10. Specifically, the gasket 200 can be sized and shaped to surround the sheet of filter material 102 or to overlay the perimeter 101 of the sheet of filter material 102. As shown in FIGS. 2A-2B, the gasket 200 can have an outer edge 202, an inner edge 204, an upper surface 208 and a lower surface 209 both extending from the outer edge 202 to the inner edge 204. The gasket 200 can have a height H extending from the upper surface 208 to the lower surface 209, i.e. a height of the outer edge 202 and inner edge 204. The gasket 200 can have a width W extending from the outer edge 202 to the inner edge 204, i.e. a width of the top upper surface 208 and lower surface 209. The outer edge 202 can be configured to align with an outer edge of the seal rim 17 of the base 12, as shown in FIG. 1. The inner edge 204 can define an enclosed open space 206 having generally the same size and dimensions as the upper opening 18 of the base 12. The height H can be shorter than or about equal to the height L of the lip 26 of the lid 24. For example, the height H can be in a range from about 0.25 inches (0.63 cm) to about 2 inches (6.1 cm), such as from about 0.5 inches (1.25 cm) to about 1.5 inches (3.81 cm), for example from about 0.75 inches (1.91 cm) to about 1.25 inches (3.18 cm), or any range or value therebetween.

As best illustrated in FIGS. 2B and 3, the gasket 200 can be configured to provide quick and easy detection of defects, such as rips, tears, or holes, to ensure the integrity of the seal of the sterilization container 10 during sterilization and storage post-sterilization. For example, in one embodiment as illustrated in FIG. 2B, the gasket 200 can include two layers that have a high color contrast or intensity difference in order to facilitate breach detection. FIG. 2B shows a cross-sectional view of the gasket 200 taken along the line 2-2, where the gasket 200 can be made from an inner core 210 and an outer layer 212. The inner core 210 can have a red, orange, bright green, or any other visibly bright color. The outer layer 212 can have a black or any other visibly dark color. In other words, the inner core 210 and the outer layer 212 can have a sufficient level of color contrast so that a breach (e.g., puncture, tear, rip, or hole) of the outer layer 212 of the gasket 200 can be easily detected since the inner core 210 can be visible through the breach of the outer layer 212. The outer layer 212 can be very thin such that any cut, tear, hole, rip, or other defect in the gasket 200 will expose the bright color of the inner core 210, thereby indicating that the gasket 200 is damaged or defective. The outer layer 212 can be in a range from about 15% to about 80% of the total thickness or height H of the gasket 200. As such, depending on the total thickness or height H of the gasket 200, the outer layer 212 can have a thickness in a range from about 0.03 inches to about 1.5 inches, such as from about 0.05 inches to about 1 inch, for example from about 0.1 inches to about 0.5 inches, or any range or value therebetween. For example, FIG. 2B illustrates a gasket 200 having a red-colored inner core 210 and a very thin black outer layer 212. As shown in FIG. 3, various breaches 222 in the outer layer 212 of gasket 200 are detected by the visibility of the red inner core 210.

In this regard, the term “contrast” means differences in appearance that are visually distinct to the naked eye, such as color differences, hue or value differences, tint or color saturation differences, opacity differences, translucence differences, and differences related to the ability to see through articles. For instance, differences between similar colors can amount to a contrast if they demonstrate a color difference or distance between two colors, referred to by the Commission Internationale de l'Eclairage (CIE) as the ΔE* value, greater than about 2.3, where it is generally known that a ΔE* of 2.3 corresponds with a just noticeable color difference. Specifically, the color difference between the outer layer 212 and the inner core 210, in terms of the ΔE* value, can be greater than about 2.5, such as greater than about 3, such as greater than about 3.5, where the L*a*b* color value measurements, which refer to a sample's luminosity value (L*), red to green color difference value (a*), and yellow to blue color difference value (b*), and ΔE* calculations (CIE 1976 Commission Internationale de l'Eclairage) may be made using an X-Rite 938 Spectrodensitometer D65/10° using CMY filters, in accordance with the operator's manual, or any other suitable device. The X-Rite Spectrodensitometer may be obtained from the X-Rite Corporation of Grandville, Mich. Average optical densities are generally taken as the sum of the average of three measurements using each filter ΔE* is calculated in accordance with the following equation:

ΔE*=SQRT[(L*standard−L*sample)2+(a*standard−a*sample)2+(b*−standard−b*sample)2]

Where L* represents lightness (0=black and 100=white). Further, the color channels, a* and b* will represent true neutral gray values at a*=0 and b*=0. The red/green opponent colors are represented along the a* axis, with green at negative a* values and red at positive a* values. Meanwhile, the yellow/blue opponent colors are represented along the b* axis, with blue at negative b* values and yellow at positive b* values. The higher the ΔE* value, the greater the change in color intensity. Testing may be conducted in accordance with ASTM DM 224-93; ASTM D2244-15a; and/or ASTM E308-90, or any other suitable method known by one of ordinary skill in the art. A detailed description of spectrodensitometer testing is available in Color Technology in the Textile Industry, 2nd Edition, Published 1997 by AATCC (American Association of Textile Chemists & Colorists).

The CIE L*c*h* color model can also be used to analyze the difference between two colors. Essentially it is in the form of a sphere. There are three axes; L*, c* and h°. The L* axis represents Lightness and is the vertical axis. L values can range from 0 at the bottom, which represents no lightness (i.e. absolute black) through 50 in the middle, to 100 at the top, which represents maximum lightness (i.e. absolute white). The c* axis represents chroma or saturation. This ranges from 0 at the center of the circle, which represents color that is completely unsaturated (i.e., a neutral grey, black or white) to 100 or more at the edge of the circle, which represents colors that have a very high chroma (saturation) or color purity. The h* axis represents hue. If a horizontal slice is taken through the center of the sphere, cutting the ‘sphere’ (‘apple’) in half, we see a colored circle. Around the edge of the circle we see every possible saturated color, or hue. This circular axis is known as h° for hue. The units are in the form of degrees° (or angles), ranging from 0° (red) through 90° (yellow), 180° (green), 270° (blue).

Taking the color models discussed above into account, the present inventors have found that the specific combination of components present in the inner core 210 can result in a layer that is a bright color without the layer exhibiting any “bleed out.” Specifically, the inner core can exhibit sufficient levels of saturation and value so that the inner core appears bright and vivid, rather than washed out or overly dark. The saturation or chroma refers to the purity of a color. As shown in FIG. 4, on a scale of 0% to 100%, a high saturation % refers to a color that appears rich and full, while a low saturation % refers to a color that appears washed out, dull, and grayish. Referring to FIG. 4, as the amount of pure color decreases, the saturation % drops. For example, the colors shown on the top row in the chart of FIG. 4 have saturation of 100% and have no white, while the colors shown on the bottom row of the chart in FIG. 4 have a saturation of 0% and have high levels of white. In other words, as colors get very low in saturation % and approach 0% saturation, they become pastels. Meanwhile, value refers to the lightness or darkness of a color. As shown in FIG. 5, a low value % refers to a color with high levels of black, while a high value % refers to a color with no black. For example, the colors shown on the top row in the chart of FIG. 5 have a value of 100% and have no black, while the colors shown on the bottom row in the chart of FIG. 5 have a value of 0% and have high levels of black. In other words, as the rows of colors move down the chart the value % decreases, where more black is added until each color is essentially black.

As mentioned above, due to the specific components of the inner core 212 and the ratios at which they are present, the inner core 212 of the gasket 200 of the present invention is able to exhibit a saturation level greater than about 25%, such as a saturation level greater than about 30%, such as a saturation level greater than about 40%, such as a saturation level greater than about 50%, such as a saturation level greater than about 60%, such as a saturation level greater than about 70%. For instance, in some preferred embodiments, the saturation level can range from about 50% to about 100%, such as from about 60% to about 100%, such as from about 70% to about 100%, such as from about 80% to about 100%. Further, the inner core of the gasket of the present invention is able to exhibit a value level greater than about 25%, such as a value level greater than about 30%, such as a value level greater than about 40%, such as a value level greater than about 50%, such as a value level greater than about 60%, such as a value level greater than about 70%. For instance, in some preferred embodiments, the value level can range from about 50% to about 100%, such as from about 60% to about 100%, such as from about 70% to about 100%, such as from about 80% to about 100%. In addition, although it is preferred that the saturation and value levels be greater than about 25%, such levels may be more difficult to achieve. As such, saturation and value levels of less than about 25%, such as less than about 20%, such as less than about 15% are also contemplated in some embodiments of the present invention.

In addition, as a result of the specific components of each of the gasket layers (e.g., the outer layer 212 and the inner core 210) and the processing conditions by which the gasket is made, the gasket layers can have sufficient color contrast without “bleeding” or “muddying” of the darker color associated with one of the layers through the other lighter colored layer. For example, the inner core 210 can include a lighter colored pigment (e.g., red, orange, yellow, green, blue, indigo, violet, or a combination thereof), whereas the outer layer 212 can include a darker colored pigment (e.g., black, brown, dark gray, blue, purple, etc.). Additionally, the inner core 210 can include a titanium dioxide or similar filler in combination with the color pigment to provide a desired level of color, contrast, brightness, saturation, value, and/or opaqueness. The present inventors have discovered that the ratio of the parts of titanium dioxide to the colored pigment in the inner core 210 can be controlled to achieve an inner core having sufficient value and saturation percentages as discussed above. Referring to Table 1 below, in some embodiments, the outer layer 212 of the gasket 200 and the inner core 210 of the gasket can include the following color combinations, where possible Pantone color codes that can be used for the colorants are included in parentheses:

TABLE 1
Gasket Color Combinations

| Outer Layer | Inner Core |
|---|---|
| Black | Orange (2018U) |
| Black | Green (2286U) |
| Black | Blue (2174U) |
| Black | Red (2028U) |
| Black | Yellow (102U) |
| Black | Pink (238U) |
| Grey (435U) | Purple (265U) |
| Grey (435U) | Orange (2018U) |
| Grey (435U) | Green (2286U) |
| Grey (435U) | Red (2028U) |
| Grey (435U) | Blue (2174U) |
| Grey (435U) | Yellow (102U) |
| Dark Blue (2965U) | Green (2286U) |
| Dark Blue (2965U) | Yellow (102U) |
| Dark Blue (2965U) | Orange (2018U) |
| Green (2286U) | Purple (265U) |
| Purple (265U) | Green (2286U) |
| Blue (2174U) | Yellow (102U) |
| Blue (2174U) | Orange (2018U) |

In one embodiment, the inner core 210 and outer layer 212 of the gasket 200 can be formed from a single material, such as polyurethane, silicone, polyvulcanate, polyvinylidene chloride (PVDC), polytetrafluoroethylene (PTFE), polysulphones, crosslinked elastomers, etc. However, in some embodiments, the inner core 210 can be made of a first material and the outer layer 212 can be made of a second material. For example, the first and second materials of the inner core 210 and outer layer 212 can have different properties such as their amount of compressibility. For example, in one particular embodiment, the inner core 210 can optionally provide structural support to the gasket 200. In such an embodiment, the inner core 210 can be formed from a more rigid polymer such as non-foamed polyurethane, silicone, polyvulcanate, polyvinyl chloride (PVC), polyvinylidene chloride (PVDC), polyvinylidene fluoride (PVDF), polytetrafluorethylene (PTFE), crosslinked and mineral filled elastomers, and other hard durometer materials, while the outer layer 212 can be formed from a more compressible material such as a foam, including foamed polyurethane, foamed silicones, foamed polyvinyl chloride (PVC), foamed elastomers, foamed polyvinylidene chloride (PVDC), polyvinylidene chloride (PVDF), and other soft durometer polymers, so that the gasket 200 is sufficiently compressible to adequately seal the lid 24 to the base 12 at the seal rim 17. In another embodiment, the inner core 210 and the outer layer 212 can be formed from the same base material but with different compression properties. For example, the inner core 210 and the outer layer 212 can be the same polymer with two different durometers, where outer layer 212, which is the material closer to the interface with the seal rim 17, has a lower durometer and is thus more compressible than the inner core 210.

Further, the gasket 200 can be disposable or reusable. A disposable gasket can reduce the risk of wear and tear, which can result in inadequate sealing capabilities, by requiring that the gasket 200 is single-use or so relatively inexpensive to the consumer that it can be discarded if a breach is detected or it becomes otherwise unusable after only one or a few uses. Alternatively, the inner core 210 and/or outer layer 212 of the gasket 200 can be formed from a reusable material that is more durable and can withstand multiple sterilization cycles without losing its compressibility. Suitable materials can include elastomeric silicone, polytetrafluoroethylene, polyvinylidene fluoride, polyurethane, and/or a polyolefin (e.g., polyethylene or polypropylene).

The material(s) of the inner core 210 and outer layer 212 of the gasket can be compounded with pigments prior to shaping in order to achieve the contrasting colors to enable breach detection as described above. Alternatively, the inner core 210 and outer layer 212 can be formed and then dyed with respective pigments.

In one embodiment, the inner core 210 can be formed in the shape and dimensions of the final gasket 200, i.e. having an outer edge 202 of generally the same dimensions as the outer edge of the seal rim 17 of the base 12 and an inner edge 204 of generally the same dimensions as the upper opening 18 of the base 12. The inner core 210 can be made by molding, extrusion, or other suitable method of forming polymers.

Molding according to the present invention is a process of manufacturing by shaping pliable raw material using a rigid frame called a mold or matrix. The mold or matrix itself may have been made using a pattern or model of the final object (i.e., the inner core 210 or gasket 200). The mold or matrix can be a hollowed-out block that is filled with the pliable material, i.e. the material of the inner core 210 of the gasket 200. The pliable material hardens in the mold, adopting its shape, sometimes after having a specific temperature and/or pressure applied. Generally, any molding process which is known to the skilled person can be used in the present invention.

Examples of molding processes are, but not limited to, blow molding, compaction plus sintering, compression molding, expandable bead molding, extrusion molding, foam molding, injection molding, reaction injection molding, matched mold, matrix molding, pressure plug assist molding, rotational molding, transfer molding, thermoforming, vacuum plug assist molding, and the like. After applying a specific temperature and pressure to the pliable material in the mold for a specific period of time, the final product (e.g., the inner core 210 or the fully formed gasket 200) can be obtained. The skilled person will be aware of the process conditions in order to obtain a product having the desired properties. In one embodiment a release agent may be used to make removal of the inner core 210 and/or gasket 200 from the mold easier.

After the formation, e.g. molding or extrusion, of the inner core 210, then the outer layer 212 can be formed in a very thin layer onto the already formed inner core 210. For example, the outer layer 212 can be overmolded onto the inner core 210. Alternatively, the inner core 210 and outer layer 212 can be formed by co-extrusion, room temperature casting, or any other suitable method of forming the outer layer 212 over the inner core 210 of the gasket 200.

In one embodiment, the inner core 210 and outer layer 212 can be co-extruded. Any known type of extruders can be used in the present invention to form the inner core 210 and/or outer layer 212, such as, but not limited to, ram extruder, screw extruder, double screw extruder, planetary-gear extruder and cascade extruder, or the like. The extruder may be chosen depending on the materials of the inner core 210 and outer layer 212 of the gasket 200. Co-extrusion in the sense of the present invention can mean that the inner core 210 and outer layer 212 are extruded separately through different die openings (nozzles) of the same extruder and are processed afterwards, i.e. the materials of the inner core 210 and outer layer 212 are not mixed before reaching the die openings of the extruder but can be processed together after being co-extruded.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

What is claimed is:

1. A sealing assembly for a sterilization packaging system, the sealing assembly comprising:
a gasket having an outer edge, an inner edge, and an upper surface and a lower surface each extending from the outer edge to the inner edge,
wherein the gasket comprises an outer layer and an inner core,
wherein the outer layer includes a first material compounded with a first colored pigment, and the inner core includes a second material compounded with a second colored pigment,
wherein a sufficient level of contrast exists between the outer layer and the inner core to detect a breach of the outer layer.

2. The sealing assembly of claim 1, wherein a breach of the outer layer exposes the second colored pigment of the inner core to facilitate detection of the breach.

3. The sealing assembly of claim 1, wherein the outer layer is darker than the inner core, wherein the outer layer and the inner core exhibit a ΔE* color difference greater than about 2.5 as determined according to the Commission Internationale de l'Eclairage (CIE) 1976 standard.

4. The sealing assembly of claim 1, wherein the inner core exhibits a saturation level of greater than about 25%.

5. The sealing assembly of claim 1, wherein the inner core exhibits a value level of greater than about 25%.

6. The sealing assembly of claim 1, wherein the second material is less compressible and more rigid than the first material.

7. The sealing assembly of claim 1, wherein the first material and the second material are made from the same polymer.

8. The sealing assembly of claim 7, wherein the first material and the second material have different durometers, wherein the first material has a lower durometer than the second material.

9. The sealing assembly of claim 1, wherein the first material and the second material are made from polyurethane, silicone, polyvulcanate, polyvinylidene chloride (PVDC), polytetrafluoroethylene (PTFE), polysulphones, crosslinked elastomers, or a combination thereof.

10. A sterilization packaging system having a volume for containing items to be sterilized, the sterilization packaging system comprising:
a lid having an upper surface defining a perimeter and a lip extending downward from the perimeter;
a base having a lower surface, a first sidewall, a second sidewall, a third sidewall, and a fourth sidewall extending from the lower surface, wherein the first sidewall, the second sidewall, the third sidewall, and the fourth sidewall terminate at a seal rim defining a perimeter of an opening of the base;
a filter; and
a sealing assembly, wherein the sealing assembly is configured to seal the lid to the seal rim of the base, the sealing assembly comprising:
a gasket having an outer edge, an inner edge, and an upper surface and a lower surface each extending from the outer edge to the inner edge,
wherein the gasket comprises an outer layer and an inner core,
wherein the outer layer includes a first material compounded with a first colored pigment, and the inner core includes a second material compounded with a second colored pigment,
wherein a sufficient level of contrast exists between the outer layer and the inner core to detect a breach of the outer layer.

11. The sterilization packaging system of claim 10, wherein a breach of the outer layer of the gasket exposes the second colored pigment of the inner core of the gasket to facilitate detection of the breach.

12. The sterilization packaging system of claim 10, wherein the outer layer of the gasket is darker than the inner core of the gasket, wherein the outer layer of the gasket and the inner core of the gasket exhibit a ΔE* color difference greater than about 2.5 as determined according to the Commission Internationale de l'Eclairage (CIE) 1976 standard.

13. The sterilization packaging system of claim 10, wherein the inner core of the gasket exhibits a saturation level of greater than about 25%.

14. The sterilization packaging system of claim 10, wherein the inner core of the gasket exhibits a value level of greater than about 25%.

15. The sterilization packaging system of claim 10, wherein the second material of the gasket is less compressible and more rigid than the first material of the gasket.

16. The sterilization packaging system of claim 10, wherein the first material of the gasket and the second material of the gasket are made from the same polymer.

17. The sterilization packaging system of claim 16, wherein the first material of the gasket and the second material of the gasket have different durometers, wherein the first material has a lower durometer than the second material.

18. The sterilization packaging system of claim 10, wherein the first material and the second material are made from polyurethane, silicone, polyvulcanate, polyvinylidene chloride (PVDC), polytetrafluoroethylene (PTFE), polysulphones, crosslinked elastomers, or a combination thereof.

19. The sterilization packaging system of claim 10, wherein the sealing assembly is configured to provide a continuous sealing interface between the lid and the base.

20. The sterilization packaging system of claim 10, wherein the sealing assembly and the filter are disposable, further wherein the lid and the base are reusable.

21. The sterilization packaging system of claim 10, wherein the sealing assembly is reusable and the filter is disposable, further wherein the lid and the base are reusable.
